(19) United States
(12) Patent Application Publication
(10) Pub. No.: US 2009/0076362 A1
(43) Pub. Date: Mar. 19, 2009

(54) Electrode Structure

(76) Inventor: Jukka Jaatinen, Kempele (FI)

Correspondence Address: HOFFMANN & BARON, LLP, 6900 JERICHO TURNPIKE, SYOSSET, NY, 11791, US

(21) Appl. No.: 11/857,489

(22) Filed: Sep. 19, 2007

(51) Int. Cl. A61B 5/0408

(52) U.S. Cl. 600/372

(57) ABSTRACT

An electrode structure includes at least one pair of electrode units, and at least one electrode unit including a fabric component for a skin contact. The fabric component is electrically connectable to an electric circuit of a user-specific performance monitor system.

### BACKGROUND OF THE INVENTION

1. Field of the Invention

The invention relates to an electrode structure, a measuring unit, a user-specific monitoring system including the electrode structure and a method of manufacturing them.

2. Description of the Related Art

A heart rate measurement device is an example of a user-specific performance monitor system. When the heart beats, it causes a series of electric impulses that can be measured from the body. The measurement and analysis of the signal is called electrocardiography (ECG), and the signal itself is known as an ECG signal. Different phases of the heart cycle can be distinguished in the signal.

A heart rate measurement device comprises a measurement belt fastened around the chest. The belt has metallic electrodes for measuring the electric pulses of the heart of the user. The belt may include a wireless transmitter that transmits a signal associated with the pulses of the heart. The user may have a separate watch-like monitoring device around the wrist as a receiver. The monitoring device comprises a measuring unit that may contain, for example, an electronic signal processing unit for processing the ECG signal, a display, a user interface, and a wristband for attaching the measuring device to the users hand. The monitoring device processes the received signal and shows a piece of information on the heart rate to the user.

The heart rate can be measured from the user's hands without the belt around the chest using only a measuring device similar to the monitoring wristwatch. The inner surface of the measuring device that is set against the hand may be provided with one or more metallic electrodes, each one of which acts as an electrode and is in contact with the skin. The measuring unit further comprises a second metallic pad as an electrode on the outer surface of the measuring device. The electrodes are connected to the signal processing unit of the measuring unit. To produce a contact with the electrodes, the user must touch it with one finger of his/her other hand. With each hand thus in contact with a separate electrode, the signal processing unit is capable of measuring and processing the ECG signal transmitted by the electrodes.

However, this kind of measurement involves a number of problems. The contact of the electrodes to the skin should be better and the shape of the electrodes should follow that of the skin. The metallic electrodes against the skin may also irritate the skin.

### SUMMARY OF THE INVENTION

An object of the invention is to provide an improved electrode structure. According to an aspect of the invention, there is provided an electrode structure of a skin contact for a user-specific performance monitor system, the electrode structure comprising at least one pair of electrode units, at least one electrode unit comprising a fabric component for a skin contact, the fabric component being of an electrically isolating material and the fabric component being electrically connectable to an electric circuit of the user-specific performance monitor system.

According to another aspect of the invention, there is provided a measuring unit comprising an electrode structure of a skin contact and a transmitter, the electrode structure comprising at least one pair of electrode units, at least one electrode unit comprising a fabric component for a skin contact, the fabric component being of an electrically isolating material and the fabric component being electrically connectable to the transmitter configured to transmit a signal associated with a signal detected by the at least one pair of electrode units.

According to another aspect of the invention, there is provided a user-specific performance monitor system comprising an electrode structure of a skin contact, the electrode structure comprising at least one pair of electrode units, at least one electrode unit comprising a fabric component for a skin contact, and the fabric component being of electrically isolating material and the fabric component being an electrically connectable to an electric circuit of the user-specific performance monitor system.

According to another aspect of the invention, there is provided a method of manufacturing an electrode structure of a skin contact for a user-specific performance monitor system, the method comprising forming an electrode structure with at least one pair of electrode units; and forming at least one electrode unit with a fabric component for a skin contact, the fabric component being of an electrically isolating material and the fabric component being electrically connectable to an electric circuit of the user-specific performance monitor system.

The invention provides several advantages. Fabric electrodes are smooth and provide a good contact to the skin. The electrodes also follow the shape of the skin well. A fabric electrode does not irritate even a sensitive skin so easily.

### BRIEF DESCRIPTION OF THE DRAWINGS

In the following, the invention will be described in greater detail with reference to the embodiments and the accompanying drawings, in which

FIG. 1 shows a user-specific performance monitor with separate devices;

FIG. 2 shows a user-specific performance monitor in a single device;

FIG. 3 shows two electrodes of a user-specific performance monitor;

FIG. 4 illustrates an electrode structure with a conductive electrode and an insulating electrode;

FIG. 5 illustrates a fabric component and an electrically conductive element;

FIG. 6 illustrates a fabric component and a moisture shield;

FIG. 7 illustrates a fabric component covered by an electrically conductive layer and a moisture shield layer; and

FIG. 8 illustrates a flow chart of the manufacturing method.

### DETAILED DESCRIPTION OF THE PREFERRED EMBODIMENTS

With reference to an embodiment shown in FIG. 1, a user-specific performance monitor system 150 may comprise two parts. A measuring unit 100 which may be like a belt with a transmitter 1000 may be placed around the chest of a user. A signal associated with a signal measured by the electrodes of the measuring unit 100 may be wirelessly transmitted from the belt 100 to a receiver 102, which may be implemented as a wristband worn on the wrist of the user. The transmitted signal may carry, for instance, ECG information. The location of the receiver is, however, not restricted to the wrist but may be chosen freely, provided that the wireless communication between the belt 100 and the receiver 102 is possible and the user is capable of operating the receiver 102.

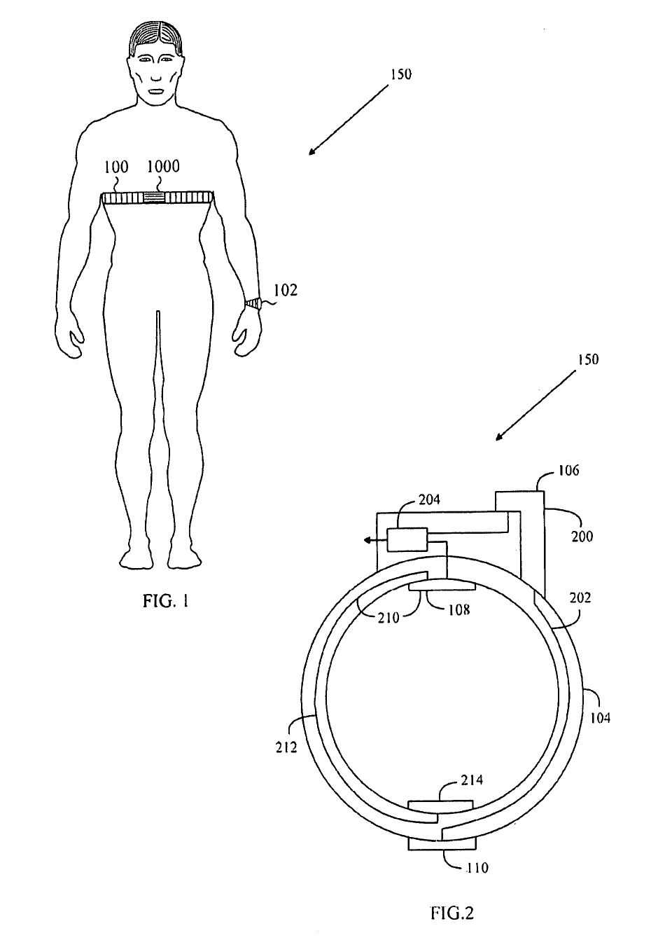

With reference to an embodiment shown in FIG. 2, the user-specific performance monitor system 150 may comprise only one part, which may partly be similar to the receiver 102 worn on the wrist of the user. In this example, the user-specific performance monitor system 150 may lack the receiving operation but may comprise an outer electrode unit 200 connected to a signal processor 204. The electrode unit 200 located on the outer surface of the system may comprise at least two electrodes 106, 110, which do not necessarily cover the whole outer surface, but instead the electrodes 106, 110 may form areas separated from each other by an insulating outer surface and the wristband.

The outer electrode unit 200 may, however, comprise a conductor 202 inside the wristband 104, and the conductor 202 may connect the electrodes 106, 110 together. The user-specific performance monitor system may further comprise an inner electrode structure 210, which is also connected to the signal processor 204. The conductive inner electrode structure 210 may also comprise a number of separate electrodes 108, 214. Accordingly, the inner electrode unit 210 may comprise at least one electrode 214 coupled directly or indirectly to the processor 204. In the case of at least two electrodes 108, 214, the electrodes 108, 214 may be coupled together with the conductor 212 inside the wristband 104 and to the processor 204. The conductors 202, 212, may be proper electrical wires or made of some electrically conductive material, such as a conductive polymer.

The signal processor 204 may measure the heart rate from signals received from the outer and inner structures. From the signal processor 204 a measured signal may then be supplied in a wireless or wired transmission to a computer, for example. The signal processor 204 may be implemented using separate logic components or one or more ASIC circuits (Application Specific Integrated Circuit), for example.

Without the connection established via the skin and the signal processor 204, there is no galvanic contact between the outer electrode unit 200 and the inner electrode unit 210, but they are electrically isolated from one another. They may be separated by plastic or some other electrically isolating or poorly conductive material of the wristband 104.

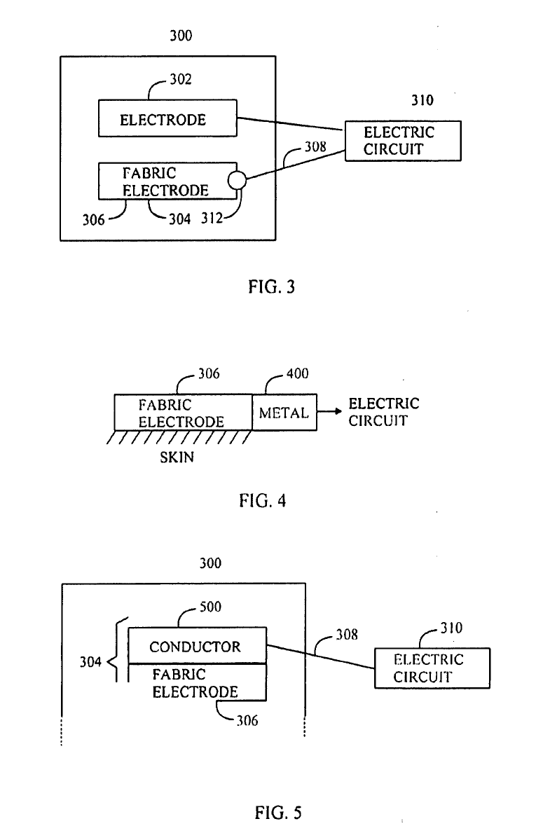

Let us now examine an electrode structure of a skin contact for a user-specific performance monitor system a little bit closer. FIG. 3 presents the electrode structure 300 comprising a pair of electrode units 302, 304, but the electrode structure 300 may also comprise more than one pair of the electrode units. In an embodiment, at least one of the electrode units 302, 304 may comprise a fabric component 306 for the skin contact which readily adapts to the skin surface. As an example, in FIG. 3 the electrode unit 304 is made of fabric. In general, the at least one electrode unit 302, 304 may be fully or partly fabric. The at least one fabric component 306 may be, for example, felt, cloth, textile or tissue. The fabric may be made of a thread of natural or man-made fibers. Furthermore, the fabric may be woven, non-woven or knitted and the fabric may comprise organic or non-organic fibers.

The fabric, per se, may be electrically isolating, but when it has absorbed a suitable fluid, electric current may flow through it. The fabric may be treated, for instance, with usual (tap) water, water with ions (such as salty water), sweat, gel, oil, fat, any combination of these, etc. The substances that may be used to moisten the fabric component 306 may act as an electrolyte in the fabric component 306 and hence provide an electrically conductive medium for enabling a good conductivity in the fabric. A water column and/or other liquid-like substance(s) reaching from the surface of the skin into the fabric component 306 also improve(s) the contact.

The user may, before a performance, moisten the fabric component 306 with water or he/she may expect the sweat on the skin to moisten the fabric component 306. The user may also apply some gel, oil, fat or a desired substance to the fabric component 306. To absorb water well, the fabric component 306 may be hygroscopic.

The at least one fabric component 306 may, in turn, be electrically connected to an electric circuit 310 of a user-specific performance monitor system with a conductor 308. The fabric component 306 may be fastened to the electrode structure 300 and coupled to the conductor 308 with, for example, at least one press-stud or some other quick-disconnect fitting 312.

The fabric component 306 may be light, flexible and elastic. The fabric component 306 may feel soft and it is adaptable on the skin. Various materials can be used for the fabric component so as to take into account sensitiveness and allergies of different users. The fabric component can be cleaned simply by washing.

As shown in FIG. 4, at least one electrode unit 304 may comprise an electrically conductive element 400 which is conductive, per se, without treating it with a liquid-like substance. In FIG. 3 the electrode unit 302 is drawn to be fully made of an electrically conductive material, such as metal or a conductive polymer. But the electrically conductive element 400 may also only be a portion of the electrode unit 304 without the skin contact.

FIG. 5 presents an embodiment where at least one electrically conductive element 500 is configured to convey the signal from the fabric component 306 towards the electric circuit 310 of the user-specific performance monitor system. When the fabric component 306 is in contact with the skin and receives an electrical signal from the body, the signal is transferred through the fabric component 306 treated with the fluid to the electrically conductive element 500. As the electrically conductive element 500 and the electric circuit 310 of the user-specific performance monitor system 310 are connected to each other by a conductor 308, the signal can propagate from the electrically conductive element 500 to the system 310.

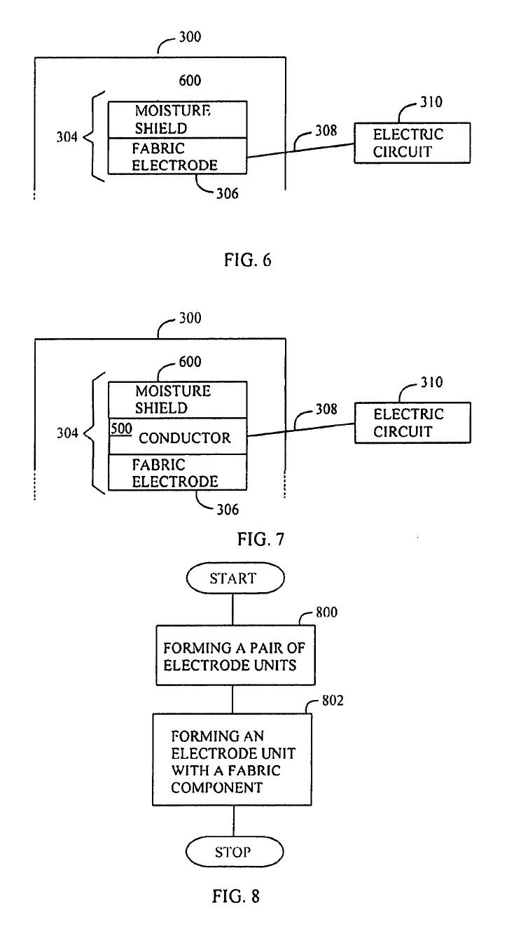

FIG. 6 presents an embodiment where the electrode unit 304 comprises a moisture shield 600 on each fabric component 306. The moisture shield 600 may be made of a waterproof or non-respiring fabric or a film-like material, such as plastic, silicone plastic or rubber. The moisture shield 600 may be constructed as a laminate of suitable materials. The moisture shield may retain moisture in the contact area of the fabric component 306. The moisture shield may be a layer that is impervious, and the moisture shield 600 may prevent the evaporation of moisture from the fabric component 306 and hence keep the fabric component 306 moistened and conductive during performance.

FIG. 7 illustrates an embodiment where the electrode unit 304 comprises an electrically conductive element 500 and a moisture shield 600 on each fabric component 306. The electrically conductive element 500 may be connected to the electric circuit of the user-specific performance monitor system using a conductor 308.

As it can be understood from the figures above, the electrode structure may comprise layers in a sandwich structure such that a layer of the fabric component 306 is covered with at least one layer such as an electrically conductive element 500, a moisture shield 600 or both. However, the electrode structure may also have another construction than that of a sandwich structure.

FIG. 8 illustrates a method of manufacturing the electrode structure. In step 800, an electrode structure with at least one pair of electrode units is formed. In step 802, at least one electrode unit with a fabric component for a skin contact is formed, the fabric component being electrically connectable to an electric circuit of a user-specific performance monitor system. The fabric component may have been woven of threads or just compressed of fibers.

Even though the invention has been described above with reference to an example according to the accompanying drawings, it is clear that the invention is not restricted thereto but it can be modified in several ways within the scope of the appended claims.

What is claimed is:

1. An electrode structure of a skin contact for a user-specific performance monitor system, the electrode structure comprising at least one pair of electrode units, at least one electrode unit comprising a fabric component for a skin contact, the fabric component being of an electrically isolating material and the fabric component being electrically connectable to an electric circuit of a user-specific performance monitor system.

2. The electrode structure of claim 1, wherein at least one electrode unit comprises an electrically conductive element.

3. The electrode structure of claim 2, wherein at least one electrically conductive element is configured to convey a signal from the fabric component towards the electric circuit of the user-specific performance monitor system.

4. The electrode structure of claim 1, wherein the at least one electrode unit comprises a moisture shield on each fabric component.

5. The electrode structure of claim 1, wherein the fabric component is hygroscopic.

6. The electrode structure of claim 1, wherein the electrode structure is a layered structure such that a layer of the fabric component is covered with at least one layer.

7. The electrode structure of claim 6, wherein the at least one layer on the fabric component is a layer of a electrically conductive element.

8. The electrode structure of claim 6, wherein the at least one layer on the fabric component is a layer of a moisture shield.

9. The electrode structure of claim 6, wherein the fabric component is covered with a layer of an electrically conductive element layer and the layer of the electrically conductive element is covered with a layer of a moisture shield.

10. The electrode structure of claim 1, wherein the fabric component is configured to be treated with a fluid to become electrically conductive for a performance.

11. The electrode structure of claim 10, wherein the fluid is at least one of the following: water, sweat, gel, oil, fat.

12. A measuring unit comprising:
an electrode structure of a skin contact; and
a transmitter, the electrode structure comprising at least one pair of electrode units, at least one electrode unit comprising a fabric component for a skin contact, the fabric component being of an electrically isolating material and the fabric component being electrically connectable to the transmitter configured to transmit a signal associated with a signal detected by the at least one pair of electrode units.

13. The measuring unit of claim 12, wherein at least one electrode unit comprises an electrically conductive element.

14. The measuring unit of claim 13, wherein at least one electrically conductive element is configured to convey a signal from the fabric component towards the electric circuit of the user-specific performance monitor system.

15. The measuring unit of claim 12, wherein the at least one electrode unit comprises a moisture shield on each fabric component.

16. A user-specific performance monitor system comprising an electrode structure of a skin contact, the electrode structure comprising at least one pair of electrode units, at least one electrode unit comprising a fabric component for a skin contact, and the fabric component being of an electrically isolating material and the fabric component being electrically connectable to an electric circuit of the user-specific performance monitor system.

17. The user-specific performance monitor system of claim 16, wherein at least one electrode unit comprises an electrically conductive element.

18. The user-specific performance monitor system of claim 17, wherein at least one electrically conductive element is configured to convey a signal from the fabric component towards the electric circuit of the user-specific performance monitor system.

19. The user-specific performance monitor system of claim 16, wherein the at least one electrode unit comprises a moisture shield on each fabric component.

20. A method of manufacturing an electrode structure of a skin contact for a user-specific performance monitor system, the method comprising:
forming an electrode structure with at least one pair of electrode units; and
forming at least one electrode unit with a fabric component for a skin contact, the fabric component being of an electrically isolating material and the fabric component being electrically connectable to an electric circuit of the user-specific performance monitor system.
